(19) United States
(12) Patent Application Publication
(10) Pub. No.: US 2008/0187457 A1
(43) Pub. Date: Aug. 7, 2008

(54) Antibacterial Titanium Dioxide Compositions

(76) Inventor: John R. Mangiardi, Greenwich, CT (US)

Correspondence Address: HUNTON & WILLIAMS/NEW YORK;INTELLECTUAL PROPERTY DEPT., 1900 K STREET, N.W., SUITE 1200, WASHINGTON, DC, 20006-1109, US

(21) Appl. No.: 11/937,102

(22) Filed: Nov. 8, 2007

Related U.S. Application Data
(60) Provisional application No. 60/888,175, filed on Feb. 5, 2007.

(51) Int. Cl. A61L 2/10; B05D 3/06; C08F 2/46

(52) U.S. Cl. 422/24; 422/22; 427/553; 522/66

(57) ABSTRACT

An acrylic or other polymer composition, optionally containing aluminum trihydrate, and having undoped or doped titanium dioxide dispersed throughout is disclosed. Said composition provides a “renewable” surface and further provides a highly-sterile surface upon photoactivation of the surface by at least either low-level ultraviolet irradiation or ambient/natural light (as when using doped titanium dioxide).

### CROSS-REFERENCE TO RELATED APPLICATIONS

This application claims the benefit of provisional application U.S. 60/888,175, filed Feb. 5, 2007, entitled “ANTIBACTERIAL TITANIUM DIOXIDE COMPOSITIONS” and the complete content of this application is incorporated by reference.

### BACKGROUND OF THE INVENTION

1. Field of Invention

The present invention relates to compositions comprising particles of titanium dioxide dispersed throughout and a method of activating the germicidal properties of said titanium dioxide by photoactivation.

2. Background of the Invention

Titanium dioxide is a naturally occurring form of oxidized titanium and has found many industrial applications. It is cheap, widely available, and has been recognized by the U.S. Food and Drug Administration as harmless to humans. It is most commonly recognized as a white pigment in paints or cosmetics. Less well known are its photocatalytic properties. When added to coating materials such as paint or when placed on surfaces such as windows, it breaks down organic compounds and acts as both a sterilizer and deodorizer. This activity is greatly enhanced by exposure to ultraviolet radiation.

Acrylic polymers containing aluminum trihydrate are solid surface materials that have demonstrated useful properties. Typically, such compounds are inert, nontoxic and hypoallergenic. Additionally, such compounds are non-porous and because they do not absorb materials, will limit bacterial or fungicidal growth.

### SUMMARY OF THE INVENTION

The present invention is, in one or more embodiments, an antibacterial and antifungal surface material comprising a polymer having dispersed throughout: an amount of TiO₂ and/or doped TiO₂ effective to result in at least the surface of said solid surface material becoming at least 99% sterile upon exposure to at least one of the following: ambient, natural, or ultraviolet light conditions. The present invention is also a method of using the above solid surface material, wherein a) objects or areas in an environment are surfaced by the surface material; b) said surface materials in the environment are periodically irradiated with at least one of the following: i) ultraviolet light; or ii) natural or ambient light; and c) said surface material is resurfaced when the surface of said composition is damaged. Finally, the present invention is also, in one or more embodiments, a method of creating a sterilizable environment from an environment comprising a) surfacing said environment with a surface material having dispersed throughout a compound which reacts in the presence of ambient, natural, or ultraviolet light to sterilize at least the surface of said surface material such that exposure results in the surface becoming at least 99% sterile.

In one embodiment, the present invention comprises the synergistic coupling of two compositions (a polymer and a photoactivated catalyst) to provide a new composition with enhanced properties. The composition may be adjoined with yet another composition to provide additional functionality and further improved qualities. Titanium dioxide, alone, or dispersed on a surface, is not a useful composition for antibacterial activities because over time the dispersion is dissipated through normal wear and tear. As such, the useful half-life of titanium dioxide is low and there is a present need for a composition in which the useful properties of titanium dioxide and its derivatives or cognate groups are extended.

Acrylic (or other similar) polymers are useful but like any solid material composition can harbor bacteria or other contaminants. While relatively less conducive to bacterial growth, acrylic polymers alone are insufficient for environments where a high sterility is demanded. For example, hospital operating rooms or home or restaurant kitchens demand higher sterility. While a dispersion of titanium dioxide on such a surface might provide antibacterial properties, the effective half-life of such a combination is low. As the surface becomes damaged or scratched, lacunae in the surface develop which can harbor bacteria. Owing to the general property that acrylics (and other similar polymers) can be sanded or resurfaced, this invention comprises a polymeric composition (acrylic in a preferred embodiment) that contains titanium dioxide or titanium dioxide containing compounds dispersed throughout the polymer. Such dispersal is preferably homogenous or “even” in that the photocatalytically active species are generally equidistant from one another. However, the dispersal must only be effected in a manner that ensures that the sufficient compound is present on the surface after a given number of surface renewals that the material retains a useful amount of sterilizing capability.

This invention further discloses that photoactivation of the composition by irradiation with ultraviolet light (and in some embodiments, by normal natural light or ambient light such as from an artificial source like an incandescent bulb) greatly enhances the sterilizing properties of the composition. As such, a high sterility environment can be maintained by periodic irradiation of the environment. Further, unlike other compositions that utilize “antibacterial” compounds, titanium dioxide in a polymeric composition is environmentally safe. Therefore, biohazard creation is minimized or obviated, especially during sanding during which time fine particulates might be created. As such, the present invention is at least, in one or more embodiments: a) an environmentally safe and low-cost composition which provides a sterilizable and renewable surface that provides enhanced sterility under light conditions; and b) a method of creating a sterile environment and of renewing the sterility of that environment.

### DETAILED DESCRIPTION OF THE INVENTION

In a preferred embodiment, titanium dioxide, preferably in the anatase form is added to a composition of acrylic polymer (or other polymer composition) containing aluminum trihydrate in an amount effective to ensure complete sterility of the surface when exposed to, at least, low level ultraviolet radiation for an effective time. In a preferred and exemplary embodiment, the titanium dioxide is added to provide a composition containing from 0.1 to 1% titanium dioxide in the anatase form by volume. In another embodiment, the titanium dioxide may be present up to about 1% titanium dioxide but not greater than 1.1%. While these ranges are preferred, other concentration amounts may also be used provided that the concentration is not so great as to cause flaking of the material or clouding of the surface. Concentrations below 1.1% meet this requirement but greater concentration levels are contemplated. Further, in applications where clouding and/or flaking are not a concern, higher concentrations may be added to optimize the sterility feature of the invention.

The titanium dioxide as dispersed is thereby also effective to maintain sterility after an initial first sterilization for a reasonable period of time before a second or repeated sterilization is initialized, such as prior to an event requiring high sterility. The acrylic or other polymer composition containing titanium dioxide can be placed as a surface material such as in a hospital room. Owing to the unique properties of acrylic polymers containing aluminum trihydrate, the surface material can be molded and set such that seams or crevices are obviated. Other polymers having such molding attributes are also preferred. Thus, a method of providing a sterile working environment is achieved. The room's major surfaces are comprised of this improved composition and periodically irradiated to maintain high sterility. High sterility is defined as a surface being at least 99% free of bacteria or fungi as determined by, for example, a culture test.

In an alternative embodiment, the composition remains substantially the same but nitrogen (or carbon or boron) doped TiO₂ is used instead. Nitrogen doped TiO₂ is preferred. The addition of nitrogen allows the anatase form of TiO₂ to become photocatalytically active in normal or ambient light, i.e. UV light is not required to activate sterilization. Such doped titanium compounds are described by Chang, et al. in Applied and Environmental Microbiology, 9/2006, p 6111-6116 and by Han, et al. in the Journal of Zhejiang University SCIENCE B, 2005 7(4):299-303, both of which are hereby incorporated by reference. Dispersion of such materials throughout a composition, e.g. an acrylic or other polymer, would provide a solid surface material of profound benefits.

Continuing, as the surface of the improved composition is damaged or scratched, the solid surface material can be sanded, polished, or otherwise renewed by removal of a portion of the outer layer to provide a fresh, uniform surface with renewed antibacterial and other properties. Such resurfacing could be done on a periodic basis to ensure that high sterility is maintained. For example, the composition could be sanded once a month, once a year, or at other periodic intervals.

In an alternative embodiment of the present invention, a surface material that is non-translucent is provided. Such a material retains the sterility properties of the main embodiments of the invention because TiO₂ is dispersed throughout. Such a material can be used in alternate settings, such as school desktops, shower stalls, flooring, sinks, walls, and other surfaces where translucence to light is not required but sterility is. In either the main or alternative embodiments, the materials can be used in healthcare environments, homes, schools, restaurants, hotels, and other commercial, residential, and industrial facilities. The dispersion of TiO₂ can also be in a substance, which will harden into a solid surface such as a lacquer or unhardened acrylic. In such a case, the material can be poured, such as on a floor, allowed to harden, and thereby provide a thick, sterile coat. Such a coat can be translucent or colored by the addition of pigments and is layered or poured in sufficient volume that the hardened surface can be renewed at least one or more times by sanding, polishing, or other resurfacing methods.

It should be noted that in all instances where sterilization occurs, the materials and compositions of the present invention are adapted—by dispersion of an effective amount of photocatalyst, e.g. titanium dioxide or doped titanium dioxide—to provide a sterile surface under ultraviolet irradiation in under a day under normal ambient temperature and preferably under a day with only ambient, natural light under normal ambient temperatures and in at most under a week under the same conditions. Provided a direct light source (UV or otherwise) the sterilization may occur much faster. Tests have determined that surface materials made in accordance with at least one or more of the embodiments of the present invention, are sterilizable in as little as an hour under appropriate lighting conditions. Stronger wattage light sources provide faster sterilization times. Certain wavelengths of light perform better and can be determined by routine experimentation. Direct lighting, as opposed to lighting of a surface at an oblique angle, provides optimal sterilization times. Greater concentrations of titanium dioxide improve sterilization times but it is a teaching of the present invention that concentrations above 1.1% are to be avoided in certain applications, particularly when the transparency and integrity of the material is vital. Finally, references to the surface of the surface material are to be construed as indicating the exposed surface of the material, e.g. sterilization of the surface of the surface material indicates that at least the exposed surface of the surface material is sterilized. While any portion of the surface may be sterilized, only the exposed surface of the material will usually receive adequate light exposure. The exposed surface is also the most critical area for sterilization.

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention. It is further known that other modifications may be made to the present invention, without departing the scope of the invention, as noted in the appended claims.

I claim:

1. A surface material comprising
a polymer having dispersed throughout an effective amount of TiO₂ and/or doped TiO₂ to result in at least the surface of said surface material becoming at least 99% sterile upon exposure to at least one of the following: ambient, natural, or ultraviolet light conditions.

2. The surface material of claim 1 in which at least the surface of said surface material becomes at least 99.99% sterile upon exposure to at least one of the following: ambient, natural, or ultraviolet light conditions.

3. The surface material of claim 1 in which any doped-TiO₂ is doped with boron, carbon, and/or nitrogen.

4. The surface material of claim 1 in which any doped-TiO₂ is doped with substances containing boron, carbon, and/or nitrogen.

5. The surface material of claim 1 in which said TiO₂ and/or doped TiO₂ is present in up to and including 1.1% by volume.

6. The surface material of claim 1 in which said polymer is an acrylic polymer comprising aluminum trihydrate.

7. The surface material of claim 1 in which said surface material is formed by hardening or allowing to harden a non-solid composition containing said TiO₂ and/or doped TiO₂ dispersed throughout.

8. The surface material of claim 7 in which said non-solid composition is a lacquer, paint, or resin.

9. The surface material of claim 1 in which said solid surface material is a panel, optionally comprising curves.

10. The panel of claim 9 in which said panel is at least 0.25 inches in thickness, at least 0.25 inches in length, and at least 0.25 inches in height.

11. The panel of claim 9 in which said panel is adapted to receive a screw, bolt, joint, clip, thread, or nail.

12. A method of using the surface material in claim 1, wherein a) objects or areas in an environment are surfaced by the surface material; b) said surface materials in the environment are periodically irradiated with at least one of the following: i) ultraviolet light; or ii) natural or ambient light; and c) said surface material is resurfaced when the surface of said composition is damaged.

13. The method of claim 12 wherein said environment is a hospital operating room, in a healthcare environment, in a home, in a school, in a restaurant, in a hotel, or in a commercial/industrial/or residential facility.

14. The method of claim 12 wherein said surface is renewed by sanding.

15. The method of claim 12 wherein said surface is renewed.

16. The method of claim 12 wherein said surface is polished.

17. A method of creating and maintaining a sterilizable environment from an environment comprising a) surfacing said environment with a surface material having dispersed throughout a compound which reacts in the presence of ambient, natural, or ultraviolet light to sterilize the surface of said surface material such that exposure results in at least the surface becoming at least 99% sterile; and b) renewing the surface of said surface material when said surface material is damaged.

18. The method of claim 17 in which the surface of said surface material is adapted to sterilize to at least 99% under exposure to light within a day.

19. The method of claim 18 in which said compound is titanium dioxide and/or doped titanium dioxide.
